(19) United States
(12) Patent Application Publication
(10) Pub. No.: US 2014/0242621 A1
(43) Pub. Date: Aug. 28, 2014

(54) METHOD FOR DETERMINING PROTEIN S ACTIVITY

(71) Applicant: Siemens Healthcare Products Diagnostics GmbH, Marburg (DE)

(72) Inventors: Juergen Patzke, Marburg (DE); Joern Meuer, Lahntal (DE); Thomas Wissel, Lahntal (DE)

(73) Assignee: Siemens Healthcare Products Diagnostics GmbH, Marburg (DE)

(21) Appl. No.: 14/178,345

(22) Filed: Feb. 12, 2014

(30) Foreign Application Priority Data
Feb. 28, 2013 (EP) 13157111.9

(51) Int. Cl. C12Q 1/56

(52) U.S. Cl. CPC C12Q 1/56
USPC 435/13; 435/326; 530/389.2

(57) ABSTRACT

The invention relates to a method for determining the protein Ca-cofactor activity of protein S, wherein the protein Ca-cofactor activity of protein S is measured in a first reaction volume in the absence of a protein S inhibitor and in a second reaction volume in the presence of a protein S inhibitor. The protein Ca-cofactor activity of protein S is determined by calculating the quotient of the assay result of the first reaction volume and the assay result of the second reaction volume.

The invention is in the field of coagulation diagnostics and relates to an in vitro method for determining protein S activity.

The blood coagulation system performs two opposing tasks: on the one hand, it uses anticoagulatory mechanisms to ensure that a continuous bloodstream is maintained; on the other hand, in the event of vascular injury, it uses procoagulatory mechanisms to ensure that wound closure takes place.

The activation of the coagulation system as a result of vascular injury ultimately gives rise to the active protease thrombin (factor IIa) via a cascade system of activating proteases. The initially only very slow thrombin formation is sped up by thrombin itself, by the thrombin activating the cofactors factor V and VIII by proteolytic cleavage. On phospholipid surfaces, the activated cofactors factor Va and VIIIa form with the proteases factor Xa and IXa an enzyme-cofactor complex which has thrombin-activating activity that is about 1000 times higher than the activity of the proteases alone. This positive feedback leads to an explosive formation of large amounts of thrombin. Thrombin converts fibrinogen to fibrin, which polymerizes and leads to wound closure.

To prevent a life-threatening escalation of the coagulation, which would lead to occlusion of the vascular system in the body, it is necessary both for the thrombin formed to be inactivated and for the formation of further thrombin to be stopped. Active thrombin is neutralized by complex formation with thrombin inhibitors such as, for example, antithrombin. The formation of further thrombin is restricted by the thrombin itself. Thrombin binds to the membrane protein thrombomodulin. The cell-associated thrombin-thrombomodulin complex activates the inhibitor protein protein C (PC) to form protein Ca (activated protein C, APC). APC inactivates the factors Va and VIIIa and thus restricts the formation of thrombin. For optimum anticoagulatory action, APC requires, inter alia, protein S as cofactor.

Protein S is a vitamin K-dependent plasma protein. In the blood, some protein S is bound to C4b-binding protein (C4b-BP). Only about 40% of the protein S present is available as unbound, free protein S. Only the free protein S stimulates the inactivation of factor Va and VIIIa via APC. This anticoagulatory activity of protein S is also referred to as protein Ca-cofactor activity or APC-cofactor activity.

In addition to the protein Ca-cofactor activity which leads to the inactivation of factor Va and VIIIa, free protein S can also directly inhibit the procoagulatory prothrombinase complex and the procoagulatory tenase complex, by binding to factor Va and to factor Xa. This second anticoagulatory activity of protein S is also referred to as APC-independent activity (van Wijnen, M. et al., A plasma coagulation assay for an activated protein C-independent anticoagulant activity of protein S. Thromb Haemost 1998, 80: 930-035).

Therefore, protein S has an important inhibitory function in the coagulation system. Protein S deficiency or reduced protein S activity are acknowledged risk factors for thrombophilia. Particularly the activity of protein S is an important marker for estimating risk of thrombosis.

Determining protein S activity in a clinical laboratory is frequently achieved by determining the protein Ca-cofactor activity of the protein S. To this end, the sample is usually mixed with a standardized amount of activated protein C, and coagulation is activated, producing factor Va which is inhibited by the added activated protein C. The protein S present in the sample acts as a cofactor for the activated protein C. The higher the protein S activity in the sample, the longer the coagulation time of the reaction volume. Known, commercially available assays are, for example, the Protein S Ac assay from Siemens Healthcare Diagnostics, Marburg, Germany or the STA Protein S clotting assay from Diagnostica Stago, France.

A problem is that various influencing variables can impair the determination of the protein Ca-cofactor activity of the protein S and can lead to false measured results.

One known influencing variable is a frequently occurring mutation of the factor V at the protein Ca cleavage site of the factor V (factor V Leiden), resulting in factor Va being resistant to activated protein C (APC-resistance), i.e., factor Va can no longer be inactivated by protein Ca. In samples from patients having an (undiagnosed) factor V Leiden mutation, protein S cannot develop its protein Ca-boosting action, the coagulation reaction proceeds unimpeded, and the protein S activity determined is therefore, incorrectly, too low. To exclude the interfering variable factor V Leiden mutation, it is proposed in the prior art that preactivated factor Va be added to the sample (Wolf, M. et al., A new functional assay for human protein S activity using activated factor V as substrate. Thromb Haemost. 1989, 62(4): 1144-1145 and STA Protein S clotting assay).

Another known influencing variable is the presence of thrombocytes in the sample. In plasma samples which, as a result of improper processing, contain more than 10 000 thrombocytes/μL, there is the risk that the protein S activity determined is, incorrectly, too low (Patzke, J. et al., Characterization of a protein S assay measuring the APC cofactor activity. J Lab Med 2007, 31(6): 262-272).

Yet another known influencing variable is antiphospholipid antibodies present in the patient's sample, particularly those which impair the in vitro coagulation time of the patient's sample (lupus anticoagulants). In samples from patients with an (undiagnosed) antiphosholipid syndrome, the coagulation time-prolonging action of the lupus anticoagulants can superimpose the coagulation time-prolonging action of protein S, and so the protein S activity determined is, incorrectly, too high.

It is an object of the present invention to improve known methods for determining the protein Ca-cofactor activity of protein S, in which methods the sample is mixed with activated protein C or a substance for activating protein C and with factor Va or a substance for directly or indirectly activating factor V to give a reaction volume and the coagulation reaction in the reaction volume is measured, in such a way that the influence of any interfering variable is eliminated, enabling the true protein S activity of the sample to be specifically determined.

An exemplary embodiment of the invention that the inventors currently consider the best way of implementing the invention is described with reference to the drawings.

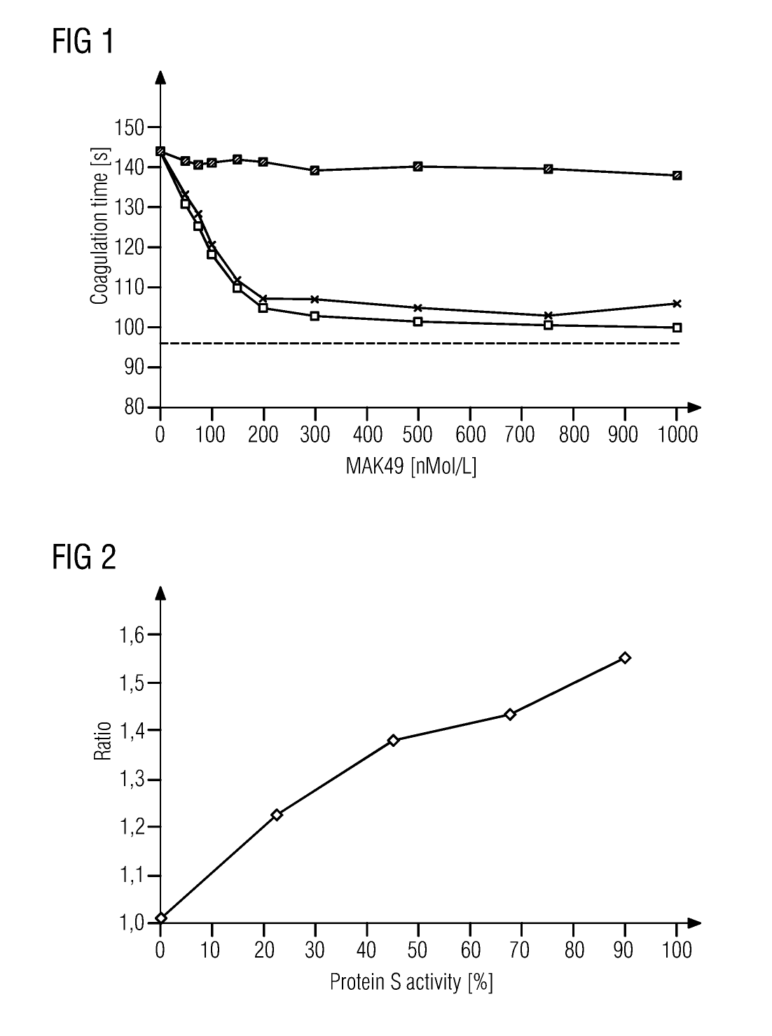

FIG. 1 shows the inhibition of the protein Ca-cofactor activity of protein S in normal plasma by the monoclonal antibody MAK49 (blank squares) compared to the protein Ca-cofactor activity of protein S in normal plasma without inhibitor (filled squares).

FIG. 2 shows a calibration curve for the measurement of samples having unknown protein S activities, in which the ratios or quotients of coagulation time measured in the absence of the MAK49 and coagulation time measured in the presence of the MAK49 are plotted against the known protein S activity of measured samples.

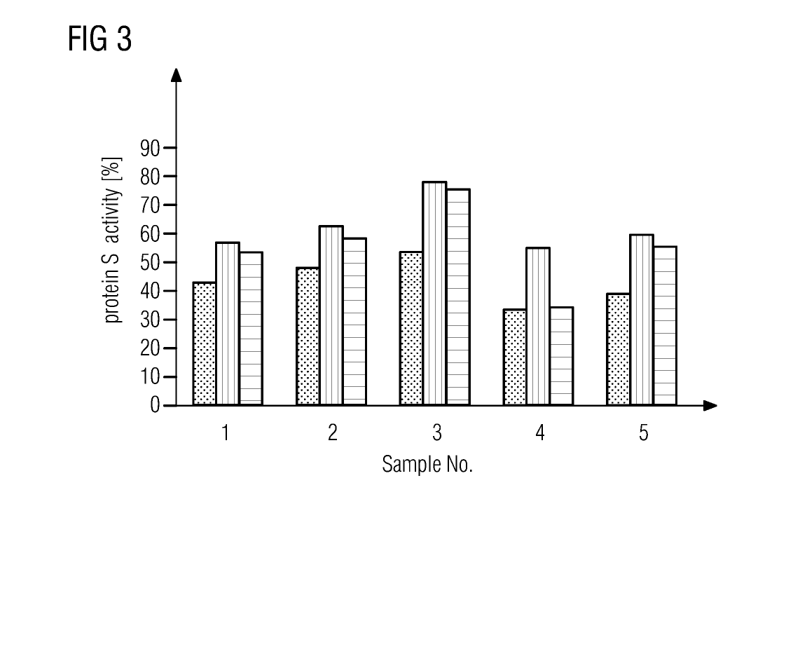

FIG. 3 shows, for five tested samples, the protein Ca-cofactor activity of protein S in the absence of the inhibitor MAK49 (dotted bars), the amount of free protein S antigen (vertically stripped bars), and the quotient of the protein Ca-cofactor activity of protein S (coagulation time) measured in the absence of the MAK49 and the protein Ca-cofactor activity of protein S (coagulation time) measured in the presence of the MAK49 (horizontally stripped bars).

The object is achieved by mixing the sample, in a second reaction volume, with an inhibitor which inhibits the protein Ca-cofactor activity of protein S, in addition to the same components which are used for providing the first reaction volume, and by measuring the coagulation reaction in the reaction volume, and then by determining the protein Ca-cofactor activity of protein S by calculating the quotient of the coagulation reaction measured in the first reaction volume and the coagulation reaction measured in the second reaction volume.

Adding to the second reaction volume an inhibitor which inhibits the protein Ca-cofactor activity of protein S causes the protein S activity in the reaction volume to be equal or close to 0%, while the influence of any interfering variables in the sample, for example factor V Leiden, excessively high thrombocyte count or antiphospholipid antibodies, on the coagulation reaction of the reaction volume remains unchanged. In the second reaction volume, only the influence of any interfering variables is thus measured. By calculating the quotient of the assay results of the first and the second reaction volume, the influence of any interfering variables is eliminated, and it is possible to quantify the true protein S activity.

An “inhibitor which inhibits the protein Ca-cofactor activity of protein S” is to be understood to mean any substance which inhibits at least the protein Ca-cofactor activity of protein S, for example monoclonal protein S-binding antibodies or protein S-binding fragments of monoclonal antibodies which were obtained by suitable, known methods for producing monoclonal antibodies and were selected according to their ability to inhibit the protein Ca-cofactor activity of protein S (see also example 1). Another suitable substance is C4b-binding protein (C4b-BP), which binds and thereby inactivates free protein S.

A preferred inhibitor which inhibits the protein Ca-cofactor activity of protein S is a monoclonal antibody which binds to protein S and which is produced by the hybridoma cell line 96-168/01. Said hybridoma cell line was deposited on Nov. 7, 2012 at the Leibniz-Institut DSMZ—Deutsche Sammlung von Mikroorganismen and Zellkulturen GmbH (Leibniz Institute DSMZ—German Collection of Microorganisms and Cell Cultures), Inhoffenstrasse 7B, 38124 Braunschweig, Germany, under the submission number DSM ACC3188. In addition, preference is given to protein S-binding fragments of the monoclonal antibody produced by the hybridoma cell line 96-168/01.

The present invention therefore further provides the hybridoma cell line deposited at the DSMZ under the submission number DSM ACC3188 and also the antibody produced by the hybridoma cell line DSM ACC 3188, and also protein S-binding fragments thereof.

For the purposes of the invention, a “sample” is to be understood to mean a material suspected of containing the protein S to be determined. The term “sample” encompasses, in particular, human or animal body fluids, principally blood and plasma, preferably low-platelet plasma.

In embodiments of the method according to the invention in which the sample is mixed with activated protein C, purified human protein Ca is preferably used. Alternatively, the sample can be mixed with a substance for activating protein C, which substance can activate the protein C in the sample and/or any protein C added to the sample. Suitable substances for activating protein C are, for example, thrombin, preferably human or bovine thrombin, or a protein C activator from snake venom, preferably from snake venom from the species Agkistrodon contortrix.

In embodiments of the method according to the invention in which the sample is admixed with factor Va, purified human or bovine factor Va or purified factor Va from rabbits is preferably used. As an alternative thereto, the sample can be mixed with a substance for directly or indirectly activating factor V. Suitable for the direct activation of factor V are, in particular, thrombin, preferably human or bovine thrombin, or a factor V activator from snake venom, preferably the factor V activator from the snake venom from the species Vipera russelli. Suitable for the indirect activation of factor V are, in particular, substances from the group of the contact phase activators, for example kaolin, silica, glass or ellagic acid, or thromboplastin. A further substance for indirectly activating factor V is a prothrombin activator, preferably the prothrombin activator from the snake venom from the species Echis carinatus. Yet a further substance for indirectly activating factor V is a factor X activator from snake venom, preferably the factor X activator from the snake venom from the species Vipera russelli.

In a further embodiment of the method according to the invention, the sample can be additionally mixed with a protein S-deficient plasma. The advantage of this is that any other factor deficiencies in the patient's sample to be examined are balanced out and have no influence on the assay result.

The coagulation reaction in the first and the second reaction volume can be measured by determining the coagulation time. To this end, the period from the time of addition of factor Va or of the substance for directly or indirectly activating factor V to the sample or to the reaction volume until the formation of a tangible fibrin clot is measured in seconds. Alternatively, a chromogenic substrate for thrombin can be used and the period from the addition of factor Va or of the substance for directly or indirectly activating factor V to the sample until the attainment of a defined rate of change in absorption can be measured.

The coagulation time can be determined by manual or automated methods. In the case of automated determination, the measurement of a mechanical or optical property of the reaction volume is appropriate, for example viscosity or turbidity. In cases of automated measurement, a property of the reaction volume is usually continuously measured and, from the time-dependent change in the property, the coagulation time as an end point can be determined with the aid of evaluation methods.

The protein Ca-cofactor activity of protein S is determined according to the invention by calculating the quotient of the coagulation reaction measured in the first reaction volume in the absence of a specific inhibitor of the protein Ca-cofactor activity of protein S, and the coagulation reaction measured in the second reaction volume in the presence of a specific inhibitor of the protein Ca-cofactor activity of protein S. The greater the quotient determined, the higher the protein Ca-cofactor activity of protein S in the sample. By comparing the quotient determined for a patient's sample with a suitable calibration curve, it is possible to determine the protein S activity.

MAK49 (horizontally stripped bars). Sample No. 4 comes from a patient who was additionally treated with an oral anticoagulant.

### EXAMPLES

### Example 1

### Production of a Monoclonal Anti-Protein S Antibody Which Inhibits the Protein Ca-Cofactor Activity of Protein S

Purified human protein S was used as immunization antigen. Six mice were each injected intraperitoneally with 20 μg of immunization antigen (protein S). After several weeks, the mice were killed, and the spleens were removed and single cell suspensions were produced. The spleen cells were fused with myeloma cells (Sp2/0), and the hybridoma cells thus obtained were transferred to microtiter cell-culture plates. The specificity of the antibodies released into the cell culture supernatant was assayed in a first step using microtiter plates coated with human protein S (coating 1 μg/ml=0.15 μg/well). An aliquot of a cell culture supernatant was pipetted into each well. After an incubation time, the supernatant was removed, and each well was washed with buffer. Subsequently, a solution containing a peroxidase-coupled anti-mouse IgG antibody was pipetted into each well. After an incubation time and washing of the wells with wash buffer, an aliquot of a chromogenic TMB solution was added to each well, and the color reaction was measured at 405 nm.

The monoclonal antibodies which were obtained after cloning and which bind specifically to protein S were subsequently tested for their ability to inhibit the protein Ca-cofactor activity of protein S. To this end, increasing concentrations of the monoclonal anti-protein S antibodies were added to normal plasma, and the protein Ca-cofactor activity of protein S was measured in the plasma samples (for measurement of the protein Ca-cofactor activity of protein S, see example 2). In parallel, purified C4b-binding protein (C4b-BP) was added to normal plasma, and the protein Ca-cofactor activity of protein S was measured.

Antibodies which make it possible to achieve inhibition of the protein S activity, which inhibition corresponds to the inhibition of the protein S activity caused by 100 nmol/L C4b-binding protein (C4b-BP) in normal plasma, are suitable as inhibitors of the protein Ca-cofactor activity of protein S. A monoclonal antibody identified in this manner (“MAK49”) is produced by the hybridoma cell line 96-168/01. Said hybridoma cell line was deposited on Nov. 7, 2012 at the Leibniz-Institut DSMZ—Deutsche Sammlung von Mikroorganismen and Zellkulturen GmbH (Leibniz Institute DSMZ—German Collection of Microorganisms and Cell Cultures), Inhoffenstrasse 7B, 38124 Braunschweig, Germany, under the submission number DSM ACC3188.

FIG. 1 shows the inhibition of the protein Ca-cofactor activity of protein S in normal plasma by the monoclonal antibody MAK49 (blank squares) compared to the protein Ca-cofactor activity of protein S in normal plasma without inhibitor (filled squares). The coagulation time was measured as described in example 2. Coagulation times of around 143 s represent 100% protein S activity, and coagulation times of around 96 s represent 0% protein S activity (protein S-deficient plasma, dashed line). Starting from a concentration of about 300 to 500 nM MAK49 in the reaction volume, maximum inhibition of the protein Ca-cofactor activity of protein S is attained. “Corrected inhibition” (asterisks) means the inhibition due solely to the presence of MAK49. The dilution-related apparent inhibition was determined by addition of corresponding amounts of buffer without MAK49 (filled squares), and the total inhibition (blank squares) was corrected by the corresponding values.

### Example 2

### Calibration of the Protein S Activity of Various Samples Having Known Protein S Activity Against the Quotient of the Coagulation Times of the Samples in the Absence and in the Presence of a Protein S Inhibitor

### Method for Determining the Protein Ca-Cofactor Activity of Protein S

To determine the protein Ca-cofactor activity of protein S in the absence or in the presence of an inhibitor which inhibits the protein Ca-cofactor activity of protein S, 95 μl of sample were mixed with 5 μl of buffer or with 5 μl of inhibitor solution (10 μM MAK49) and incubated for approximately three minutes. The samples were then diluted 1:7 with protein S-deficient plasma. 16 μL of this dilution were once again mixed with 27 μL of protein S-deficient plasma and, after incubation for 20 seconds, 58 μL of an APC reagent containing human activated protein C (APC) and calcium chloride were added. After incubation for a further 110 seconds, the coagulation reaction was started by adding 145 μL of an activator reagent containing Russell's viper venom (RVV, venom from Vipera russelli) and phospholipids for activating factor X to form factor Xa, which in turn activates factor V to form factor Va.

The protein S activity is determined according to the following principle: APC proteolytically cleaves factor Va, which arises upon activation of the coagulation cascade by RVV. Protein S acts in this process as a cofactor which considerably speeds up the reaction. This leads to a coagulation time which increases proportionally with the activity of protein S in the sample. The addition of deficient plasma ensures an adequate supply of the reaction volume with fibrinogen, factor V and the other coagulation factors required. Coagulation is initiated at the factor X stage by the factor X activator from RVV. Factor Xa forms thrombin from prothrombin, mediated by the factor Va which is still remaining. Thrombin finally converts fibrinogen.

The coagulation time was determined visually.

Plasma samples containing different protein S concentrations were produced by mixing human normal plasma having a protein S activity of 90% of the norm with protein S-deficient plasma having a protein S activity of 0% (plasma samples having 90%, 67.5%, 45%, 22.5% and 0% protein S activity), and the samples were examined using the specified method.

For each sample, the coagulation time in the absence and in the presence of the protein S-inhibiting monoclonal antibody MAK49, which inhibits the protein Ca-cofactor activity of protein S, was determined, and the quotient of the coagulation time measured in the absence of the MAK49 and the coagulation time measured in the presence of the MAK49 was calculated. The quotients thus determined were plotted against the known protein S activity of the samples, and the resulting curve was used as a calibration curve for the measurement of samples having unknown protein S activities. The calibration curve is shown in FIG. 2.

### Example 3

### Determination of the Protein Ca-Cofactor Activity of Protein S in Samples from Donors Having a Homozygote FV Leiden Mutation

Frozen plasma samples from five donors having a homozygote FV Leiden mutation were thawed in a water bath at 37° C. for 15 min, and the protein Ca-cofactor activity of protein S in the absence and in the presence of MAK49 was measured (as described in example 2). In addition, the amount of free protein S antigen was determined in each sample using an immunoassay. The results are shown in FIG. 3 in a comparative manner.

In samples from patients having a factor V Leiden mutation, protein S cannot develop its protein Ca-boosting action, the coagulation reaction proceeds unimpeded, and so the coagulation time determined is too short and the protein S activity determined is therefore, incorrectly, too low. This becomes particularly clear through the comparison with the measured protein S antigen concentration. By contrast, quotient calculation as per the method according to the invention shows a better comparability with the results of the protein S antigen determination. A single exception is a sample which comes from a homozygote factor V Leiden patient who is additionally treated with an oral anticoagulant (sample No. 4). Here, the protein S activity is equally reduced both in the protein Ca-cofactor activity assay and in the method according to the invention involving quotient calculation.

1. A method for determining the protein Ca-cofactor activity of protein S in a sample, comprising:
mixing the sample in a first reaction volume with
i. activated protein C or a substance for activating protein C and
ii. factor Va or a substance for directly or indirectly activating factor V;
measuring the coagulation reaction in the first reaction volume;
mixing the sample in a second reaction volume with an inhibitor which inhibits the protein Ca-cofactor activity of protein S, in addition to the same components which are used for providing the first reaction volume;
measuring the coagulation reaction in the second reaction volume; and
determining the protein Ca-cofactor activity of protein S by calculating the quotient of the coagulation reaction measured in the first reaction volume and the coagulation reaction measured in the second reaction volume.

2. The method as claimed in claim 1, further comprising: additionally mixing the sample in the first and the second reaction volume with protein S-deficient plasma.

3. The method as claimed in claim 1, wherein the activated protein C mixed with the sample is purified human protein Ca.

4. The method as claimed in claim 1, wherein the substance for activating protein C is thrombin or a protein C activator from snake venom, preferably from snake venom from the species Agkistrodon contortrix.

5. The method as claimed in claim 1, wherein the substance for directly activating factor V is thrombin or a factor V activator from snake venom, preferably the factor V activator from snake venom from the species Vipera russelli.

6. The method as claimed in claim 1, wherein the substance for indirectly activating factor V is a contact phase activator, preferably a contact phase activator from the group consisting of kaolin, silica, glass and ellagic acid.

7. The method as claimed in claim 1, wherein the substance for indirectly activating factor V is thromboplastin.

8. The method as claimed in claim 1, wherein the substance for indirectly activating factor V is a prothrombin activator, preferably the prothrombin activator from the snake venom from the species Echis carinatus.

9. The method as claimed in claim 1, wherein the substance for indirectly activating factor V is a factor X activator, preferably the factor X activator from the snake venom from the species Vipera russelli.

10. The method as claimed in claim 1, wherein the factor Va mixed with the sample is purified human or bovine factor Va or purified factor Va from rabbits.

11. The method as claimed in claim 1, wherein the inhibitor which specifically inhibits the protein Ca-cofactor activity of protein S is a protein S-binding antibody or a protein S-binding antibody fragment or C4b-binding protein.

12. The method as claimed in claim 11, wherein the inhibitor which specifically inhibits the protein Ca-cofactor activity of protein S is a protein S-binding antibody produced by the hybridoma cell line DSM ACC3188, or a protein S-binding fragment thereof.

13. The method as claimed in claim 1, wherein the coagulation reaction is measured in the first and the second reaction volume by determining the coagulation time.

14. A hybridoma cell line deposited at the DSMZ under the submission number DSM ACC3188.

15. An antibody produced by the hybridoma cell line as claimed in claim 14, or a protein S-binding fragment thereof.
